(19) United States
(12) Patent Application Publication
(10) Pub. No.: US 2020/0390490 A1
(43) Pub. Date: Dec. 17, 2020

(54) TREATMENT INSTRUMENT AND METHOD OF USING TREATMENT INSTRUMENT

(71) Applicant: OLYMPUS CORPORATION, Tokyo (JP)

(72) Inventors: Yuki KAWAGUCHI, Koshu-shi (JP); Tomoyuki TAKASHINO, Fuchu-shi (JP); Akinori KOBAYASHI, Hino-shi (JP)

(73) Assignee: OLYMPUS CORPORATION, Tokyo (JP)

(21) Appl. No.: 17/003,406

(22) Filed: Aug. 26, 2020

Related U.S. Application Data
(63) Continuation of application No. PCT/JP2018/007242, filed on Feb. 27, 2018.

(51) Int. Cl. A61B 18/14

(52) U.S. Cl. CPC A61B 18/1445; A61B 2017/00973

(57) ABSTRACT

A treatment instrument includes a first jaw including a first electrode and a second jaw including a second electrode. The first jaw is openable and closable, together with the first electrode, with respect to the second jaw and the second electrode. The first electrode includes a projection projecting toward the second jaw. In a base of each of the jaws, an extending surface extends from an outer edge surface of a corresponding one of the electrodes toward a side away from the projection in a width direction so as to slope away from in an opening and closing direction of the first and second jaws.

### CROSS-REFERENCE TO RELATED APPLICATIONS

This is a Continuation Application of PCT Application No. PCT/JP2018/007242, filed Feb. 27, 2018, the entire contents of which are incorporated herein by reference.

### BACKGROUND

In a known treatment instrument including a pair of openable/closable jaws, a first electrode is attached to one of the jaws (“first jaw”), and the first electrode is openable/closable together with the first jaw with respect to the other jaw (“second jaw”). A second electrode is attached to the second jaw, and the second electrode is openable/closable together with the second jaw with respect to the first jaw. During a treatment, the pair of jaws sandwiches living tissue. And heat generated by a heat generator is given as a treatment energy to the sandwiched living tissue via the first electrode. The sandwiched living tissue is then incised at the place where the projection abuts. However, it can be difficult to incise and treat the tissue appropriately using such known instruments.

### SUMMARY

The present disclosure relates generally to a treatment instrument including a pair of openable/closable jaws, and a method of using the treatment instrument.

According to one aspect, a treatment instrument including a pair of jaws including a first jaw and a second jaw, the pair of jaws being closable and openable with respect to each other and being arranged with a virtual plane interposed therebetween in a closing and opening direction of the pair of jaws. A first electrode is provided on the first jaw and is openable and closable together with the first jaw with respect to the second jaw. The first electrode includes a projection projecting toward the second jaw, and a first outer edge surface forming an outer edge of the first electrode in a width direction of the pair of jaws. The first electrode is configured to incise tissue sandwiched between the pair of jaws by the projection. A first base having a lower heat conduction rate than that of the first electrode can be provided in the first jaw on a back surface of the first electrode. The first base includes a first extending surface that extends from the first outer edge surface of the first electrode toward a side away from the projection in the width direction so as to slope away from the virtual plane in the closing and opening direction. The first extending surface in the first may abut living tissue sandwiched between the first and second jaws. A second electrode is provided on the second jaw and is openable and closable together with the second jaw with respect to the first jaw. The second electrode includes a second outer edge surface forming an outer edge of the second electrode in the width direction. A second base having a lower heat conduction rate than that of the second electrode can be provided in the second jaw on a back surface of the second electrode. The second base includes a second extending surface that extends from the second outer edge surface of the second electrode toward a side away from the projection in the width direction so as to slope away from the virtual plane in the closing and opening direction. The second extending surface in the second base can abut living tissue sandwiched between the first and second jaws.

According to another aspect, a treatment instrument includes a pair of jaw assemblies including a first jaw assembly and a second jaw assembly relatively openable and closable with respect to the first jaw assembly. A virtual plane may intersect an opening and closing direction of the pair of jaw assemblies and be located at a position where the first jaw assembly and the second jaw assembly meet each other in closed state. The first jaw assembly includes a first electrode including a projection projecting toward the second jaw assembly, and a first outer edge surface that forms an outer edge of the first electrode in the width direction of the pair of jaw assemblies. The first electrode is configured to incise living tissue sandwiched between the pair of jaw assemblies. The first jaw assembly can also include a first base that has a lower heat conduction rate than that of the first electrode and includes a first extending surface that can abut the living tissue sandwiched between the pair of jaw assemblies. The second jaw assembly includes: a second electrode including a second outer edge surface that forms an outer edge of the second electrode in the width direction; and a second base that has a lower heat conduction rate than that of the second electrode and includes a second extending surface that can abut the living tissue sandwiched between the pair of jaw assemblies. The first extending surface extends from the first outer edge surface of the first electrode toward a side away from the projection in the width direction so as to slope away from the virtual surface in the opening and closing direction. The second extending surface extends from the second outer edge surface of the second electrode in the second base toward a side away from the projection in the width direction so as to slope away from the virtual surface in the opening and closing direction.

Advantages will be set forth in the description which follows, and in part will be obvious from the description, or may be learned by practice of the disclosed exemplary embodiments. The advantages may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate exemplary embodiments, and together with the general description given above and the detailed description of the embodiments given below, serve to explain the principles of the disclosure.

FIG. 1 is a schematic diagram of a system in which a treatment instrument is used according to an exemplary embodiment.

FIG. 2 is a schematic diagram showing a configuration of a distal portion of a shaft and an end effector according to an exemplary embodiment, viewed from one side of the width direction of the end effector, with the configuration partially shown in a cross section perpendicular or substantially perpendicular to the width direction of the end effector.

FIG. 3 is a schematic diagram showing a configuration of the distal portion of the shaft and the end effector according to an exemplary embodiment, viewed from one side of the direction parallel or substantially parallel to the rotation axis of the end effector, along with the inner configuration of the shaft.

FIG. 4 is a cross-sectional view schematically showing the end effector according to an exemplary embodiment in a cross section perpendicular or substantially perpendicular to a longitudinal direction of the end effector.

FIG. 5 is a cross-sectional view schematically showing a state where living tissue is sandwiched between a pair of jaws according to an exemplary embodiment in a cross section perpendicular or substantially perpendicular to the longitudinal direction of the end effector.

FIG. 6 is a cross-sectional view schematically showing an end effector according to an exemplary embodiment in a cross section perpendicular or substantially perpendicular to a longitudinal direction of the end effector.

FIG. 7 is a cross-sectional view schematically showing an end effector according to an exemplary embodiment in a cross section perpendicular or substantially perpendicular to a longitudinal direction of the end effector.

FIG. 8 is a cross-sectional view schematically showing an end effector according to an exemplary embodiment in a cross section perpendicular or substantially perpendicular to a longitudinal direction of the end effector.

FIG. 9 is a cross-sectional view schematically showing an end effector according to an exemplary embodiment in a cross section perpendicular or substantially perpendicular in a longitudinal direction of the end effector.

FIG. 10 is a cross-sectional view schematically showing an end effector according to an exemplary embodiment in a cross section perpendicular or substantially perpendicular to a longitudinal direction of the end effector.

### DETAILED DESCRIPTION

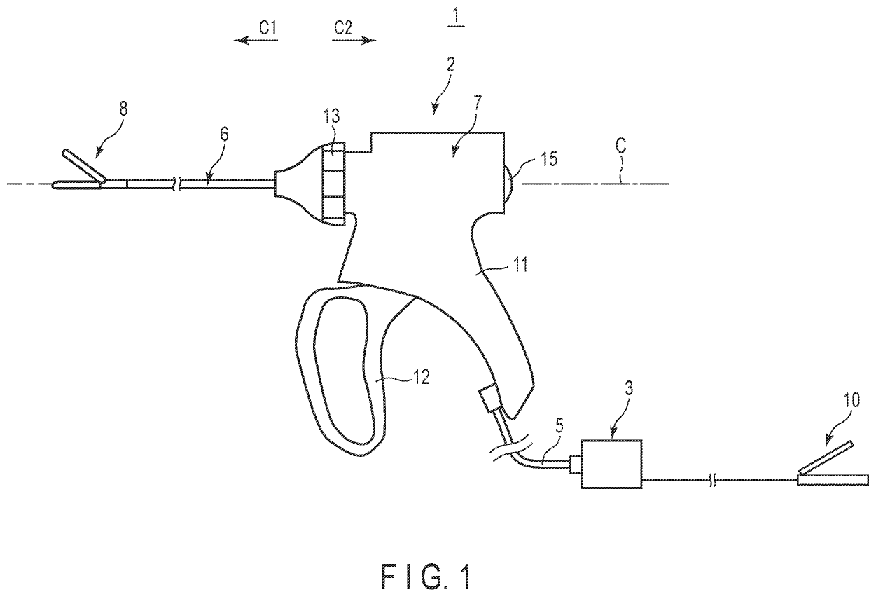

an exemplary embodiment of the present disclosure will first be described with reference to FIGS. 1 to 5. FIG. 1 is a schematic drawing of a system 1 in which a treatment instrument 2 of the present embodiment is used. As shown in FIG. 1, the system 1 includes a treatment instrument 2 and an electric power supply apparatus 3. The treatment instrument 2 is detachably connected to the electric power supply apparatus 3 via a cable 5. The treatment instrument 2 includes a cylindrical shaft (sheath) 6, a holdable housing 7 and an end effector 8. The shaft 6 extends straight or substantially straight along the longitudinal axis (central axis) C. One side of the axial direction of the shaft 6 is defined as a distal side (the side indicated by arrow C1), and a side opposite to the distal side is defined as a proximal side (the side indicated by arrow C2). The housing 7 is coupled to the proximal side of the shaft 6.

An operation apparatus 10, such as a foot switch, is electrically connected to the electric power supply apparatus 3. In the operation apparatus 10, an operation to cause the electric power supply apparatus 3 to output electric energy to the treatment instrument 2 is input. In an example, an operating button, etc. attached to a housing 7, etc. of the treatment instrument 2 is provided as an operation apparatus, instead of or in addition to the operation apparatus 10 provided separately from the treatment instrument 2. An operation to cause the electric power supply apparatus 3 to output electric energy to the treatment instrument 2 is input at the operation apparatus attached to the treatment instrument 2.

The housing 7 includes a grip 11 that extends in a direction intersecting with the longitudinal axis C. A handle 12 is pivotably attached to the housing 7. When the handle 12 rotates relative to the housing 7, the handle 12 opens or closes with respect to the grip 11. In the present embodiment, one end of the cable 5 is connected to the grip 11. The other end of the cable 5 is detachably connected to the electric power supply apparatus 3. In the present embodiment, a rotational knob 13 as an operation member is attached to the housing 7. When the rotational knob 13 is rotated with respect to the housing 7, the shaft 6 and the end effector 8 rotate around the longitudinal axis C with respect to the housing 7, together with the rotational knob 13. In one example, the rotational knob 13 is not provided, and the shaft 6 and the end effector 8 are not rotatable around the longitudinal axis C with respect to the housing 7.

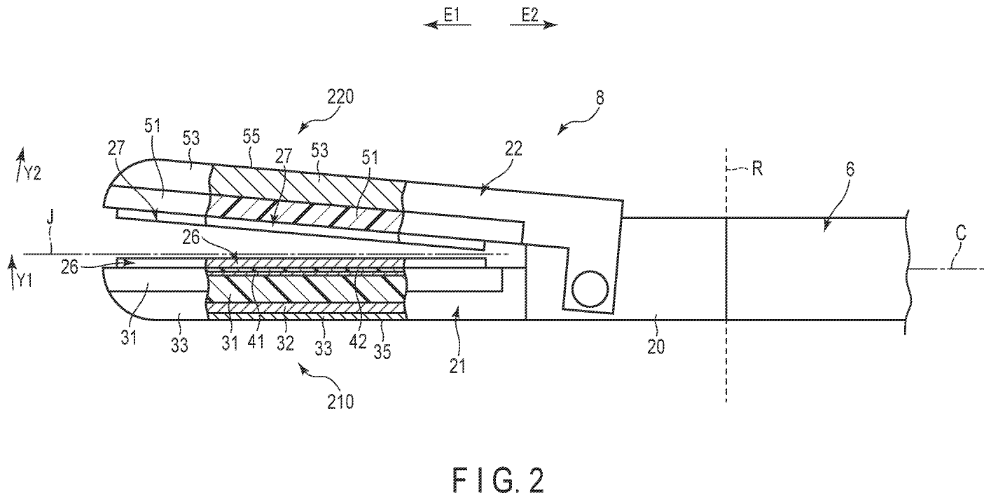

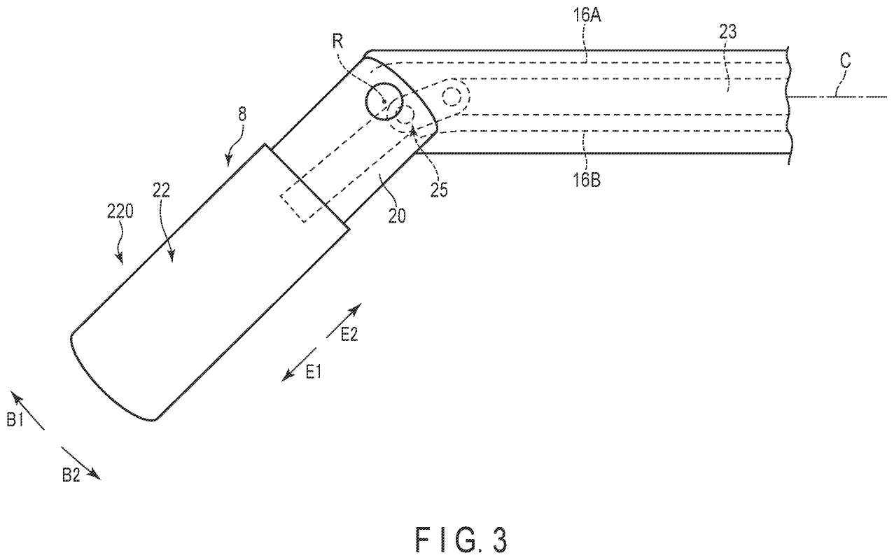

FIGS. 2 and 3 are drawings showing an example of the configuration of the distal portion of the shaft 6 and the end effector 8. As shown in FIGS. 2 and 3, the end effector 8 has a proximal end and a distal end, and extends from the proximal end to the distal end along the longitudinal direction (the direction shown by arrows E1 and E2). The end effector 8 is coupled to the distal portion of the shaft 6. The end effector 8 is rotatable about the location where it is coupled to the shaft 6, in other words, about the rotation axis R, with respect to the shaft 6. When the end effector 8 rotates around the rotation axis R with respect to the shaft 6, the end effector 8 is bent with respect to the shaft 6 and the longitudinal axis C. In a state where the end effector 8 is not bent with respect to the shaft 6, the longitudinal direction of the end effector 8 is parallel or substantially parallel to the axis direction of the shaft 6, and to the longitudinal axis C.

Herein, the rotation axis R extends in a direction intersecting with (perpendicular or substantially perpendicular to) the longitudinal direction of the end effector 8. The bending directions (the directions shown by arrows B1 and B2) of the end effector 8 intersect with (are perpendicular or substantially perpendicular to) the longitudinal direction of the end effector 8, and with the rotation axis R. Furthermore, in the present embodiment, the bending directions of the end effector 8 are parallel or substantially parallel to the width direction of the end effector 8. FIG. 2 shows the end effector 8 viewed from one side of the width direction of the end effector 8, and with the end effector 8 partially shown in a cross section perpendicular or substantially perpendicular to the width direction of the end effector 8. FIG. 3 shows the end effector 8 viewed from one side of the direction parallel or substantially parallel to the rotation axis R, along with the inner configuration of the shaft 6.

In the present embodiment, an operation dial 15 is attached as an operation member to the housing 7. A pair of long members 16A and 16B extends along the longitudinal axis C, namely the axis direction of the shaft 6. The distal end of each of the long members 16A and 16B is connected to the end effector 8. When an operation is input with the operation dial 15, driving force is transmitted to the long members 16A and 16B via a driving force transmitting mechanism (not shown) in the inside of the housing 7, and each of the long members 16A and 16B moves along the longitudinal axis C with respect to the shaft 6. Thus, the end effector 8 rotates about the rotation axis R, and the end effector 8 performs a bending motion with respect to the shaft 6.

The end effector 8 includes a cylindrical relaying portion 20 and a pair of jaws (grasping pieces) 21 and 22. The relaying portion 20 is pivotably attached to the distal portion of the shaft 6 in such a manner that the relaying portion 20 can rotate around the rotational axis R. The jaws 21 and 22 as a pair can close and open with respect to each other. One of the jaws 21 and 22 is pivotably attached to the relaying portion 20. In one example, the other of the jaws 21 and 22 is formed integrally with the relaying portion 20, or stationarily fixed to the relaying portion 20. In another example, the other of the jaws 21 and 22 is pivotably attached to the relaying portion 20, too. In another example, a rod member (not shown) projecting from the distal end of the relaying portion 20 toward the distal side is provided. In the rod member, the portion projecting from the relaying portion 20 constitutes the other of the jaws 21 and 22.

In the present embodiment, the opening and closing directions (the directions indicated by arrows Y1 and Y2) of the jaws 21 and 22, in other words, the moving directions of the jaws 21, 22 in the opening and closing motions of the end effector 8, intersect with (are perpendicular or substantially perpendicular to) the longitudinal direction of the end effector 8, and intersect with (are perpendicular or substantially perpendicular to) the bending directions of the end effector 8. The opening and closing directions of the jaws 21 and 22 (the opening and closing directions of the end effector 8) are parallel or substantially parallel to the rotation axis R.

In the inside or the outside of the shaft 6, a driving member 23 extends along the longitudinal axis C, in other words, in the axis direction of the shaft 6. The proximal portion of the driving member 23 is coupled to the handle 12 in the inside of the housing 7. The distal end of the driving member 23 is connected to the end effector 8 via a link mechanism 25. The link mechanism 25 thus connects the end effector 8 to the driving member 23. With the link mechanism 25 provided, the end effector 8 bends relative not only to the shaft 6 but also to the driving member 23.

By opening or closing the handle 12 with respect to the grip 11, the driving member 23 moves along the axis direction of the shaft 6. Thus, driving force from the driving member 23 is transmitted to the end effector 8 via the link mechanism 25, and the jaws 21 and 22 close or open with respect to each other. It is possible to grasp living tissue, etc. between the jaws 21 and 22 when they are closed with respect to each other. Furthermore, even in a state where the driving member 23 is located at any location according to the axis direction of the shaft 6, the shaft 6 is coupled to the end effector 8 within the range in which the link mechanism 25 extends. In other words, even in a state where the driving member 23 is located at any location in the direction along the longitudinal axis C, the location at which the end effector 8 is coupled to the shaft 6 is located within the range in which the link mechanism 25 extends.

In one example, the end effector 8 is not bendable with respect to the shaft 6. In this case, the operation dial 15 and the long members 16A and 16B are not provided, and the longitudinal direction of the end effector 8 is always parallel or substantially parallel to the longitudinal axis C of the shaft 6. In this example, one of the jaws 21 and 22 is pivotably attached to the shaft 6. The other of the jaws 21 and 22 may be formed integrally with the shaft 6 or stationarily fixed to the shaft 6. The other of the jaws 21 and 22 may also be pivotably attached to the shaft 6. In another example, a rod member (not shown) projecting from the distal end of the shaft 6 toward the distal side is provided. The portion in the rod member projecting from the shaft 6 constitutes the other of the jaws 21 and 22. In this example, the link mechanism 25 is not provided, and the distal end of the shaft 6 is directly connected to the end effector 8.

In the jaw 21 of the present embodiment, the dimension in the longitudinal direction of the end effector 8 is significantly larger than the dimension in the opening and closing directions of the end effector 8 and the dimension in the width direction of the end effector 8. Similarly, in the jaw 22, the dimension in the longitudinal direction of the end effector 8 is greatly larger than the dimension in the opening and closing directions of the end effector 8 and the dimension in the width direction of the end effector 8. Furthermore, in the present embodiment, the dimension of the jaw 21 in the longitudinal direction of the end effector 8 is the same or substantially the same as the dimension of the jaw 22 in the longitudinal direction of the end effector 8. The dimension of the jaw 21 in the width directions of the end effector 8 is the same or substantially the same as the dimension of the jaw 22 in the width direction of the end effector 8.

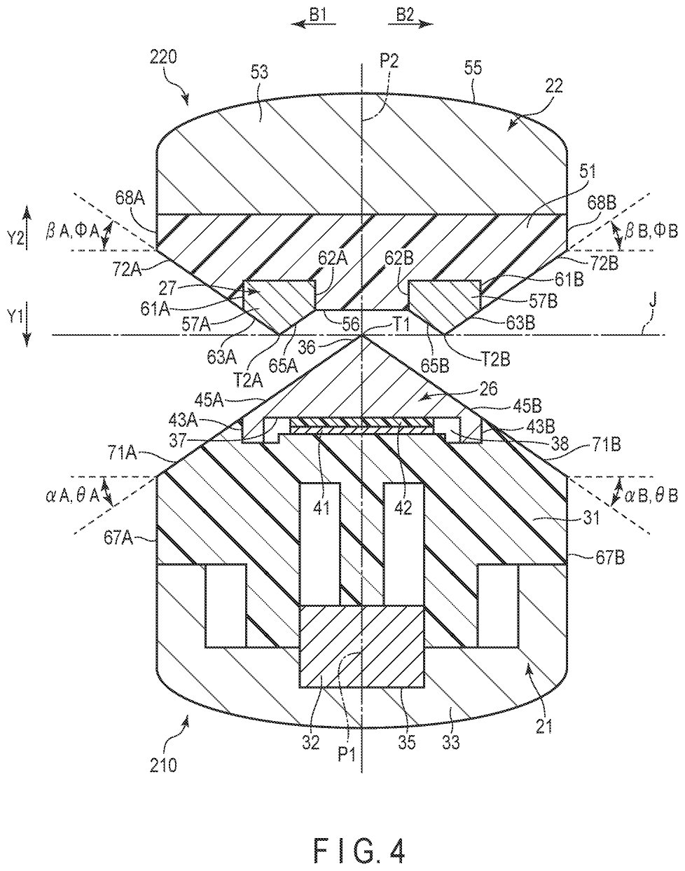

FIG. 4 shows a configuration of the end effector 8. FIG. 4 shows a cross section perpendicular or substantially perpendicular to the longitudinal direction of the end effector 8. As shown in FIGS. 2 through 4, the end effector 8 includes a pair of jaw assemblies 210 and 220. The jaw assembly (first jaw assembly) 210 and the jaw assembly (second jaw assembly) 220 can open and close with respect to each other. The first jaw assembly 210 includes at least an electrode (first electrode) 26 and a base (first base) 31. The second jaw assembly 220 includes at least an electrode (second electrode) 27 and a base (second base) 51. In the end effector 8, the pair of jaws 21 and 22 is arranged with a virtual plane J being interposed therebetween in the opening and closing directions of the jaws 21 and 22. The virtual plane J intersects with (is perpendicular or substantially perpendicular to) the opening and closing directions of the jaws 21 and 22. In the present embodiment, the virtual plane J is parallel or substantially parallel to the longitudinal direction of the end effector 8, and to the width direction of the jaws 21 and 22. The virtual plane J intersects with (is perpendicular or substantially perpendicular to) the opening and closing directions of the pair of jaw assemblies 210 and 220. The virtual plane J is located at a closed position where the pair of jaw assemblies 210 and 220 are closed to each other. Each of the jaws 21 and 22 continuously extends in a range from the proximal portion to the distal portion in the longitudinal direction of the end effector 8.

The electrode (first electrode) 26 is attached to one jaw (first jaw) 21 from the side where the other jaw (second jaw) 22 is located. The electrode 26 is openable and closable together with the jaw 21, with respect to the jaw 22. The electrode 26 continuously extends in a range from the proximal portion to the distal portion of the jaw 21 in the longitudinal direction of the end effector 8. The electrode (blade) 26 has electric conductivity and a high heat conduction rate. The electrode 26 is made of an aluminum alloy or a metal containing aluminum. The electrode (second electrode) 27 is attached to the jaw (second jaw) 22 from the side where the jaw (first jaw) 21 is located. The electrode 27 is openable and closable together with the jaw 22, with respect to the jaw 21. The electrode 27 continuously extends in a range from the proximal portion to the distal portion of the jaw 22 in the longitudinal direction of the end effector 8. The electrode 27 is made of a metal having electric conductivity, for example.

The jaw (first jaw) 21 includes a base (first base) 31, a heat sink 32, and a frame (first frame) 33. Each of the base 31, the heat sink 32, and the frame 33 continuously extends in the range from the proximal portion to the distal portion of the jaw 21 in the longitudinal direction of the end effector 8. In the jaw 21, the heat sink 32 is attached to the base 31 from the side toward which the jaw 21 opens (the back surface side of the jaw 21). The frame 33 is attached to the base 31 and the heat sink 32 from the side toward which the jaw 21 opens. The frame 33 forms, on the outer surface of the jaw 21, a jaw back surface 35 facing the side toward which the jaw 21 opens. The electrode 26 is attached to the base 31 from the side toward which the jaw 21 closes, namely the side where the jaw 22 is located.

The base 31 has electrically insulating properties, and a lower heat conduction rate than that of the electrode 26. The base 31 is made of a resin for example, such as a liquid crystal polymer (LCP) or polyetheretherketone (PEEK). In contrast, the heat sink 32 has a higher heat conduction rate than that of the base 31, and transmits heat transmitted through the base 31 toward the proximal side of the jaw 21. The heat sink 32 is made of a metal, etc. having a high heat conduction rate, such as aluminum or copper, etc. The frame 33 is made of a metal. It is preferable that the exposed portion of the frame 33 including the jaw back surface 35 is coated with an electrically insulating material, or over-molded with an electrically insulating material.

The electrode 26 includes a projection 36 projecting toward the jaw (second jaw) 22. The projection 36 projects toward the side on which the jaw (first jaw) 21 closes. The projection 36 continuously extends in a range from the proximal portion to the distal portion of the jaw 21 in the longitudinal direction of the end effector 8. The projection 36 has a projecting end T1. The electrode 26 includes an electrode back surface 37 facing the side opposite to the side where the projection 36 projects. The electrode back surface 37 faces the side toward which the jaw 21 opens, and is not externally exposed. The base 31 is attached to the electrode back surface 37 of the electrode 26, and a hollow 38 is formed between the base 31 and the electrode back surface 37 of the electrode 26. Each of the electrode back surface 37 and the hollow 38 continuously extends in the range from the proximal portion to the distal portion of the jaw 21 in the longitudinal direction of the end effector 8.

A heat generator 41, such as a heater, is provided in the hollow 38. The heat generator 41 has heater wires (not shown), and the heater wires are made of an electrically conductive material, such as stainless steel, platinum, or tungsten. The heat generator 41 is attached to the electrode back surface 37 of the electrode 26, with a bonding layer 42 interposed therebetween. The bonding layer 42 is made of a material having electric insulation properties. The heat generator 41 is electrically insulated from the electrode 26 by the bonding layer 42. Each of the heat generator 41 and the bonding layer 42 continuously extends in the range from the proximal portion to the distal portion of the jaw 21 in the longitudinal direction of the end effector 8.

Herein, a virtual central plane P1 perpendicular or substantially perpendicular to the width direction and located at the center or substantially the center of the jaw 21 according to the width direction, is defined. In the present embodiment, the virtual central plane P1 passes the projecting end T1 of the projection 36 and the heat generator 41. The jaw 21 is symmetric or substantially symmetric with respect to the virtual central plane P1. The electrode 26 is also symmetric or substantially symmetric with respect to the virtual central plane P1.

The jaw (second jaw) 22 includes a base (second base) 51 and a frame (second frame) 53. Each of the base 31 and the frame 53 continuously extends in a range from the proximal portion to the distal portion of the jaw 22 in the longitudinal direction of the end effector 8. In the jaw 22, the frame 53 is attached to the base 51 from the side toward which the jaw 22 opens. The frame 53 forms, on the outer surface of the jaw 22, a jaw back surface 55 facing the side toward which the jaw 22 opens. The electrode 27 is attached to the base 51 from the side toward which the jaw 22 closes, namely the side where the jaw 21 is located.

The base 51 has electrically insulating properties, and has a lower heat conduction rate than that of the electrode 27. The base 51 is made of a resin, for example. The frame 53 is made of a metal. It is preferable that the exposed portion of the frame 53 including the jaw back surface 55 be coated with an electrically insulating material, or over-molded with an electrically insulating material.

The base (second base) 51 includes an abutting surface 56 onto which the projection 36 of the electrode (first electrode) 26 is abuttable. The abutting surface 56 faces the projecting end T1 of the projection 36. In the present embodiment, the abutting surface 56 intersects with (is perpendicular or substantially perpendicular to) the opening and closing directions of the jaw 22. The electrode (second electrode) 27 includes a pair of electrode plates 57A and 57B. The stage 51 is attached to the back surfaces of the electrode plates 57A and 57B, namely the end surface on the side opposite to the side where the jaw 21 is located in each of the electrode plates 57A and 57B. The electrode plates 57A and 57B are arranged with the abutting surface 56 interposed therebetween, and are arranged separately from each other in the width direction of the jaw 22. For this reason, the electrode plate 57A is adjacent to one side of the abutting surface 56 in the width direction of the jaw 22. Furthermore, the electrode plate 57B is adjacent to the other side of the abutting surface 56 according to the width direction of the jaw 22. Each of the abutting surface 56 and the electrode plates 57A and 57B continuously extends in the range from the proximal portion to the distal portion of the jaw 22 in the longitudinal direction of the end effector 8. In a state where there is no living tissue between the jaws 21 and 22 and the jaws 21 and 22 are closed, the projection 36 is abutted to the abutting surface 56 in the range from the proximal portion to the distal portion of the jaw 22.

Each of the electrode plates 57A and 57B projects from the abutting surface 56 toward the jaw 21. Each of the electrode plates 57A and 57B continuously extends in the range from the proximal portion to the distal portion of the jaw 22 in the longitudinal direction of the end effector 8. The electrode plate 57A has a projecting end T2A, and the electrode plate 57B has a projecting end T2B. In a state where the projection 36 of the electrode 26 is abutted to the abutting surface 56 of the base 51, the electrodes 26 and 27 have a gap with respect to each other. For this reason, the electrode 26 and 27 are not in contact with each other.

Herein, a virtual central plane P2 perpendicular or substantially perpendicular to the width direction and located at the center or substantially the center of the jaw 22 according to the width direction, is defined. In the present embodiment, the virtual central plane P2 passes the abutting surface 56. The jaw 22 is symmetric or substantially symmetric with respect to the virtual central plane P2. In the electrode 27, the electrode plates 57A and 57B are arranged symmetrically or substantially symmetrically to each other with respect to the virtual central plane P2. In the present embodiment, the virtual central planes P1 and P2 do not deviate from each other in the width direction of the end effector 8. For this reason, the virtual central planes P1 and P2 are flush with each other.

Through an operation in the operation apparatus 10, direct current electric power or alternating current electric power is output as an electric energy from the electric power supply apparatus 3 to the heat generator 41. Heat is generated by the heat generator 41 through the supply of the electric energy to the heat generator 41. The heat generated by the heat generator 41 is transmitted to the electrode 26. In a state where living tissue, etc. is sandwiched between the jaws 21 and 22, the heat of the heat generator 41 is given as a treatment energy to the sandwiched tissue, etc. via the electrode 26.

In the present embodiment, through an operation, etc., in the operation apparatus 10, high-frequency electric power is output from the electric power supply apparatus 3 to the electrodes 26 and 27, as an electric energy different from the electric energy supplied to the heat generator 41. As a result, the electrodes 26 and 27 have electric potentials differing from each other. In a state where living tissue, etc. is sandwiched between the jaws 21 and 22, a high-frequency current flows in the tissue, etc. sandwiched between the electrodes 26 and 27. Thus, the high-frequency current is applied to the sandwiched tissue, etc. as a treatment energy via the electrodes 26 and 27.

The electrode (first electrode) 26 includes a pair of outer edge surfaces 43A and 43B, and a pair of inclined surfaces 45A and 45B. Each of the outer edge surfaces 43A and 43B and the inclined surfaces 45A and 45B continuously extends in the range from the proximal portion to the distal portion of the jaw 21 in the longitudinal direction of the end effector 8. The outer edge surfaces 43A and 43B are arranged symmetrically or substantially symmetrically to each other with respect to the virtual central plane P1. The inclined surfaces 45A and 45B are arranged symmetrically or substantially symmetrically to each other with respect to the virtual central plane P1.

The outer edge surface 43A forms an outer edge of one side of the electrode 26, according to the width direction of the jaw 21. The outer edge surface 43B forms an outer edge of the other side of the electrode 26, according to the width direction of the jaw 21. Each of the outer edge surfaces 43A and 43B faces the outward side of the width direction of the jaw 21. The outer edge surfaces 43A and 43B are parallel or substantially parallel to the longitudinal direction of the end effector 8, and parallel or substantially parallel to the opening and closing directions of the jaw 21.

The inclined surface 45A continuously extends from the projecting end T1 of the projection 36 to the outer edge surface 43A, and the inclined surface 45B continuously extends from the projecting end T1 of the projection 36 to the outer edge surface 43B. Thus, each of the inclined surfaces 45A and 45B extends outward in the width direction of the jaw 21, from the projecting end T1. Furthermore, each of the inclined surfaces 45A and 45B extends up to the outer edge surface (either 43A or 43B), toward the side away from the projecting end T1 along the virtual plane J. Each of the inclined surfaces 45A and 45B is inclined with respect to the virtual plane J, in such a manner that the surface is more separated from the jaw 22 as the surface becomes distant from the projecting end T1 in the width direction of the jaw 21. In other words, each of the inclined surfaces 45A and 45B is inclined with respect to the abutting surface 56 of the jaw 22 in such a manner that it is more separated from the jaw 22 further toward the outer side according to the width direction of the jaw 21.

The inclined surface 45A is inclined at an acute angle of αA with respect to the virtual plane J (the abutting surface 56), and the inclined surface 45B is inclined at an acute angle of αB with respect to the virtual plane J. In the present embodiment, the acute angles αA and αB are the same or substantially the same. Each of the inclined surfaces 45A and 45B is externally exposed, and faces the jaw 22. The inclined surface 45A faces the electrode plate 57A, and the inclined surface 45B faces the electrode plate 57B. Each of the outer edge surfaces 43A and 43B is not externally exposed except for the locations where it intersects with the inclined surface (either 45A or 45B) and the vicinity thereof.

In the electrode (second electrode) 27, the electrode plate 57A includes an outer edge surface 61A, an inner edge surface 62A, and inclined surfaces 63A and 65A, and the electrode plate 57B includes an outer edge surface 61B, an inner edge surface 62B, and inclined surfaces 63B and 65B. Each of the outer edge surfaces 61A and 61B, the inner edge surfaces 62A and 62B, and the inclined surfaces 63A, 63B, 65A and 65B continuously extends in the range from the proximal portion to the distal portion of the jaw 22 in the longitudinal direction of the end effector 8. The outer edge surfaces 61A and 61B are arranged symmetrically or substantially symmetrically to each other with respect to the virtual central plane P2. Similarly, the inner edge surfaces 62A and 62B are arranged symmetrically or substantially symmetrically to each other with respect to the virtual central plane P2. The inclined surfaces 63A and 63B are arranged symmetrically or substantially symmetrically to each other with respect to the virtual central plane P2. Similarly, the inclined surfaces 65A and 65B are arranged symmetrically or substantially symmetrically to each other with respect to the virtual central plane P2.

The outer edge surface 61A forms the outer edge of the electrode plate 57A in the width direction of the jaw 22, and the inner edge surface 62A forms the inner edge of the electrode plate 57A in the width direction of the jaw 22. The outer edge surface 61B forms the outer edge of the electrode plate 57B in the width direction of the jaw 22, and the inner edge surface 62B forms the inner edge of the electrode plate 57B in the width direction of the jaw 22. The outer edge formed by the outer edge surface 61A is an outer edge of one side of the electrode 27 according to the width direction of the jaw 22. The outer edge formed by the outer edge surface 61B is an outer edge of the electrode 27 on the opposite side of the outer edge surface 61A according to the width direction of the jaw 22. Each of the outer edge surfaces 61A and 61B faces outwardly in the width direction of the jaw 22, and each of the inner edge surfaces 62A and 62B faces inwardly in the width direction of the jaw 22. The outer edge surfaces 61A and 61B and the inner edge surfaces 62A and 62B are parallel or substantially parallel to the longitudinal direction of the end effector 8, and parallel or substantially parallel to the opening and closing directions of the end effector 8.

In the present embodiment, the dimension between the outer edge surfaces 43A and 43B in the width direction of the jaw 21 is the same or substantially the same as the dimension between the outer edge surfaces 61A and 61B in the width direction of the jaw 22. For this reason, the dimension between the outer edge surface 43A and the virtual central plane P1 in the width direction of the jaw 21 is the same or substantially the same as the dimension between the virtual central plane P2 and the outer edge surface 61A in the width direction of the jaw 22. Furthermore, the dimension between the outer edge surface 43B and the virtual central plane P1 in the width direction of the jaw 21 is the same or substantially the same as the dimension between the virtual central plane P2 and the outer peripheral surface 61B in the width direction of the jaw 22.

The inclined surface 63A continuously extends from the projecting end T2A of the electrode plate 57A to the outer edge surface 61A, and the inclined surface 63B continuously extends from the projecting end T2B of the electrode plate 57B to the outer edge surface 61B. Thus, each of the inclined surfaces 63A and 63B extends from a corresponding one of the projection ends T2A and T2B toward the outside in the width direction of the jaw 22. Furthermore, each of the inclined surfaces 63A and 63B extends up to the outer edge surface (either 61A or 61B) along the virtual plane J toward the side away from the projecting end T1 of the electrode 26. Each of the inclined surfaces 63A and 63B is inclined with respect to the virtual plane J in such a manner that the surface is more away from the jaw 21 as the surface becomes further away from the abutting surface 56 in the width direction of the jaw 22. In other words, each of the inclined surfaces 63A and 63B is inclined with respect to the abutting surface 56 of the jaw 22 in such a manner that the surface is more spaced from the jaw 21 as the surface becomes closer to the outer side in the width direction of the jaw 22.

The inclined surface 63A is inclined at an acute angle βA with respect to the virtual plane J (abutting surface 56), and the inclined surface 63B is inclined at an acute angle βB with respect to the virtual plane J. In the present embodiment, the acute angles βA and βB are the same or substantially the same. Each of the inclined surfaces 63A and 63B is externally exposed, and faces the jaw 21 and the electrode 26. Each of the outer edge surfaces 61A and 61B is not externally exposed, except for the location and its vicinity where the surface intersects with the inclined surface (either 63A or 63B). Furthermore, the acute angle βA of the inclined surface 63A may be the same or substantially the same as the acute angle αA of the inclined surface 45A, or may be different from the acute angle αA. Similarly, the acute angle βB of the inclined surface 63B may be the same or substantially the same as the acute angle αB of the inclined surface 45B, or may be different from the acute angle αB.

The inclined surface 65A continuously extends from the projecting end T2A of the electrode plate 57A to the inner edge surface 62A, and the inclined surface 65B continuously extends from the projecting end T2B of the electrode plate 57B to the inner edge surface 62B. Accordingly, each of the inclined surfaces 65A and 65B extends inwardly from a corresponding one of the projecting ends T2A and T2B in the width direction of the jaw 22. Furthermore, each of the inclined surfaces 65A and 65B extends up to the inner edge surface (either of 62A or 62B) toward the side close to the projecting end T1 of the electrode 26 along the virtual plane J. In the present embodiment, the abutting surface 56 continuously extends between the inclined surfaces 65A and 65B in the width direction of the jaw 22. Each of the inclined surfaces 65A and 65B is inclined to the virtual plane J in such a manner that the surface is more away from the jaw 21 as the surface becomes closer to the abutting surface 56 in the width direction of the jaw 22. In other words, each of the inclined surfaces 65A and 65B is inclined with respect to the abutting surface 56 of the jaw 22 in such a manner that the surface is more spaced from the jaw 21 as the surface become closer to the inner side in the width direction of the jaw 22.

In the present embodiment, a pair of outer edge surfaces 67A and 67B, and a pair of extending surfaces 71A and 71B are provided on a base (first base) 31, and a pair of outer edge surfaces 68A and 68B, and a pair of extending surfaces 72A and 72B are provided on a base (second base) 51. Each of the outer edge surfaces 67A and 67B and the extending surfaces 71A and 71B continuously extends in the range from the proximal portion to the distal portion of the jaw 21 in the longitudinal direction of the end effector 8. Each of the outer edge surfaces 68A and 68B and the extending surfaces 72A and 72B continuously extends in the range from the proximal portion to the distal portion of the jaw 22 in the longitudinal direction of the end effector 8. The outer edge surfaces 67A and 67B are arranged symmetrically or substantially symmetrically to each other with respect to the virtual central plane P1, and the extending surfaces 71A and 71B are arranged symmetrically or substantially symmetrically to each other about the virtual central plane P1 (the projecting end T1 of the projection 36). Similarly, the outer edge surfaces 68A and 68B are arranged symmetrically or substantially symmetrically to each other with respect to the virtual central plane P2 (the projecting end T1 of the projection 36), and the extending surfaces 72A and 72B are arranged symmetrically or substantially symmetrically to each other about the virtual central plane P2.

The outer edge surface 67A constitutes the outer edge of the base 31 in the width direction of the jaw 21, and the outer edge surface 67B constitutes the outer edge of the base 31 on the opposite side of the outer edge surface 67A according to the width direction of the jaw 21. In the present embodiment, the outer edge surface 67A constitutes the outer edge of the jaw 21 in the width direction of the jaw 21, and the outer edge surface 67B constitutes the outer edge of the jaw 21 on the opposite side of the outer edge surface 67A in the width direction of the jaw 21. Each of the outer edge surfaces 67A and 67B faces outwardly in the width direction of the jaw 21. Each of the outer edge surfaces 67A and 67B is parallel or substantially parallel to the longitudinal direction of the end effector 8, and to the opening and closing directions of the jaw 21.

Similarly, the outer edge surface 68A constitutes the outer edge of the base 51 in the width direction of the jaw 22, and the outer edge surface 68B constitutes the outer edge of the base 51 on the opposite side of the outer edge surface 68A according to the width direction of the jaw 22. In the present embodiment, the outer edge surface 68A constitutes the outer edge of the jaw 22 in the width direction of the jaw 22, and the outer edge surface 68B constitutes the outer edge of the jaw 22 on the opposite side of the outer edge surface 68A in the width direction of the jaw 22. Each of the outer edge surfaces 68A and 68B faces outwardly in the width direction of the jaw 22. Each of the outer edge surfaces 68A and 68B is parallel or substantially parallel to the longitudinal direction of the end effector 8, and to the opening and closing directions of the jaw 22.

In the present embodiment, the dimension between the outer edge surfaces 67A and 67B in the width direction of the jaw 21 is the same or substantially the same as the dimension between the outer edge surfaces 68A and 68B in the width direction of the jaw 22. For this reason, the dimension between the virtual central plane P1 and the outer edge surface 67A in the width direction of the jaw 21 is the same or substantially the same as the dimension between the virtual central plane P2 and the outer edge surface 68A in the width direction of the jaw 22. Then, the dimension between the virtual central plane P1 and the outer edge surface 67B in the width direction of the jaw 21 is the same or substantially the same as the dimension between the virtual central plane P2 and the outer edge surface 68B in the width direction of the jaw 22.

In the present embodiment, the extending surface (first extending surface) 71A extends outwardly from the outer edge surface (first edge surface) 43A of the electrode (first electrode) 26 according to the width direction. The extending surface (second extending surface) 72A extends outwardly from the outer edge surface (second edge surface) 61A of the electrode (second electrode) 27 in the width direction. Similarly, the extending surface (third extending surface) 71B extends outwardly from the outer edge surface (third edge surface) 43B of the electrode (first electrode) 26 in the width direction, and the extending surface (fourth extending surface) 72B extends outwardly from the outer edge surface (fourth edge surface) 61B of the electrode (second electrode) 27 in the width direction. Accordingly, in the present embodiment, each of the extending surfaces 71A and 71B extends from a corresponding one of the outer edge surfaces 43A and 43B of the electrode 26 toward the side away from the projecting end T1 of the projection 36 in the width direction. And each of the extending surfaces 72A and 72B extends from a corresponding one of the outer edge surfaces 61A and 61B of the electrode 27 toward the side away from the abutting surface 56 (the projecting end T1 of the projection 36) in the width direction.

As the abutting surface 56 and the extending surfaces 72A and 72B are formed as described above, the electrode plate 57A of the electrode 27 is attached to the base 51 between the extending surface 72A and the abutting surface 56 according to the width direction of the jaw 22. And the electrode plate 57B of the electrode 27 is attached to the base 51 between the extending surface 72B and the abutting surface 56 according to the width direction of the jaw 22. Furthermore, in the present embodiment, each of the extending surfaces 71A, 71B, 72A and 72B is formed in a plane shape.

The extending surface (first extending surface) 71A continuously extends outwardly in the width direction, up to the outer edge surface 67A of the base 31, and the extending surface (third extending surface) 71B continuously extends outwardly in the width direction, up to the outer edge surface 67B. Accordingly, each of the extending surfaces 71A and 71B extends outwardly according to the width direction, from a corresponding one of the outer peripheral surfaces 43A and 43B up to a corresponding one of the outer edge surfaces 67A and 67B. In other words, each of the extending surfaces 71A and 71B extends toward the side away from the projecting end T1 of the electrode 26 along the virtual plane J, up to the outer edge surface (either one of 67A or 67B). And each of the extending surfaces 71A and 71B extends in such a manner that the surface is away from the virtual plane J as the surface becomes further away from the projection 36 (the projecting end T1) of the electrode 26 in the width direction of the jaw 21. Accordingly, each of the extending surfaces 71A and 71B is inclined to the virtual plane J in such a manner that the surface is spaced from the jaw 22 as the surface becomes more outwardly according to the width direction of the jaw 21.

The extending surface 71A is inclined at an acute angle θA with respect to the virtual plane J (the abutting surface 56 of the jaw 22), and the extending surface 71B is inclined at an acute angle θB with respect to the virtual plane J. In the present embodiment, the acute angles θA and θB are the same or substantially the same. Each of the extending surfaces 71A and 71B is externally exposed, and faces the jaw 22. In the present embodiment, the acute angle θA of the extending surface 71A is the same or substantially the same as the acute angle αA of the inclined surface 45A of the electrode 26, and the acute angle θB of the extending surface 71B is the same or substantially the same as the acute angle αB of the inclined surface 45B of the electrode 26. Accordingly, the inclined surface 45A and the extending surface 71A are formed to be flush or substantially flush with each other, and the inclined surface 45B and the extending surface 71B are formed to be flush or substantially flush with each other.

The extending surface (second extending surface) 72A continuously extends outwardly in the width direction up to the outer edge surface 68A of the base 51, and the extending surface (fourth extending surface) 72B continuously extends outwardly in the width direction up to the outer edge surface 68B of the base 51. Thus, each of the extending surfaces 72A and 72B outwardly extends according to the width direction, from a corresponding one of the outer edge surfaces 61A and 61B to a corresponding one of the outer edge surfaces 68A and 68B. In other words, each of the extending surfaces 72A and 72B extends along the virtual plane J toward the side away from the projecting end T1 of the electrode 26 (the abutting surface 56), up to the outer edge surface (either one of 68A or 68B). Each of the extending surfaces 72A and 72B extends in such a manner that the surface is away from the virtual plane J as the surface becomes further away from the abutting surface 56 (the projection 36 of the electrode 26) according to the width direction of the jaw 22. Accordingly, each of the extending surfaces 72A and 72B is inclined to the virtual plane J in such a manner that the surface is separated from the jaw 21 as the surface becomes further outward in the width direction of the jaw 22.

The extending surface 72A is inclined at an acute angle φA to the virtual plane J (the abutting surface 56 of the jaw 22), and the extending surface 72B is inclined at an acute angle φB to the virtual plane J. In the present embodiment, the acute angles φA and φB are the same or substantially the same. Each of the extending surfaces 72A and 72B is externally exposed, and faces the jaw 21. In the present embodiment, the extending surface 72A faces the extending surface 71A, and the extending surface 72B faces the extending surface 71B. The acute angle φA of the extending surface 72A is the same or substantially the same as the acute angle βA of the inclined surface 63A of the electrode plate 57A (the electrode 27), and the acute angle φB of the extending surface 72B is the same or substantially the same as the acute angle βB of the inclined surface 63B of the electrode plate 57B (the electrode 27). As a result, the inclined surface 63A and the extending surface 72A are formed to be flush or substantially flush with each other, and the inclined surface 63B and the extending surface 72B are formed to be flush or substantially flush with each other.

Next, operations and advantageous effects of the treatment instrument 2 of the present embodiment will be described. When a treatment is conducted with a use of the treatment instrument 2, an operator holds the housing 7 and inserts the end effector 8 into a body cavity, such as an abdominal cavity. Then, the operator adjusts the posture of the end effector 8 by rotating it about the longitudinal axis C through the operation of the rotational knob 13, or by causing it to bend with respect to the shaft 6 through the operation of the operation dial 15. Then, the operator positions living tissue as a treatment target between the jaws 21 and 22, and closes the handle 12 relative to the grip 11. Thus, the jaws (gripping pieces) 21 and 22 are closed with respect to each other, and the living tissue is thereby sandwiched between the jaws 21 and 22.

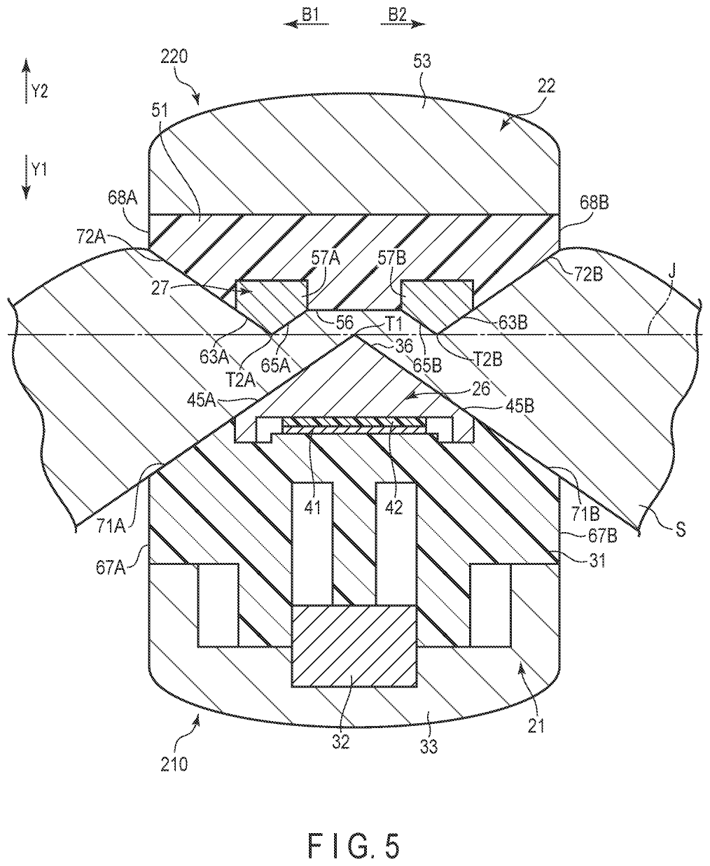

FIG. 5 is a drawing showing a state in which living tissue S is sandwiched between the jaws 21 and 22. As shown in FIG. 5, the living tissue S sandwiched between the jaws 21 and 22 is abutted to the projection 36 (the projecting end T1) and the inclined surfaces 45A and 45B in the electrode 26. The sandwiched living tissue S is abutted to the electrode plates 57A and 57B of the electrode 27. At this time the living tissue S is abutted to the electrode plate 57A on the projecting end T2A and the inclined surfaces 63A and 65A, and to the electrode plate 57B on the projecting end T2B and the inclined surfaces 63B and 65B. The sandwiched living tissue S is abutted to the extending surface 71A and 71B of the base (first base) 31. And the sandwiched living tissue S is abutted to the abutting surface 56 and the extending surfaces 72A and 72B in the base (second base) 51.

During a treatment, in a state where living tissue is sandwiched between the jaws 21 and 22 as a treatment target, an operator causes the electric power supply apparatus 3 to output a high-frequency electric power to the electrodes 26 and 27 by, for example, operating the operation apparatus 10. Thus, a high-frequency current flows between the electrodes 26 and 27 through the sandwiched living tissue, and the high-frequency current is applied to the tissue as treatment energy. Then, the tissue is sealed or coagulated by Joule heat caused by the high-frequency current. Herein, an example of the tissue sealed or coagulated using a high-frequency current is relatively thin tissue, such as blood vessels.

In a treatment for sealing or coagulating living tissue using a high-frequency current, electric energy (direct-current electric power or alternating-current electric power) different from the high-frequency electric power applied to the electrodes 26 and 27 may be output to the heat generator 41 from the electric power supply apparatus 3, in addition to the output of the high-frequency electric power to the electrode 26 and 27. In this case, in addition to the high-frequency current, the heat from the heat generator 41 is applied to the sandwiched tissue as a treatment energy, and the tissue is sealed or coagulated also by the heat generated by the heat generator 41. However, in the treatment for sealing or coagulating tissue, the temperature of the heat generator 41 is adjusted so as to fall between 100° C. and 150° C. and not to exceed 200° C.

In the present embodiment, in a state where the projection 36 of the electrode 26 is abutted to the abutting surface 56 of the base 51, the electrodes 26 and 27 have a gap with respect to each other. For this reason, in the treatment for sealing or coagulating tissue using a high-frequency current, contact between the electrodes 26 and 27 can be effectively prevented. Thus, a high-frequency current is appropriately applied to the sandwiched tissue, and the tissue is appropriately sealed or coagulated.

Furthermore, in the present embodiment, the inclined surfaces 45A and 45B are provided in the electrode (first electrode) 26, and the inclined surfaces 63A, 63B, 65A and 65B are provided in the electrode (second electrode) 27. These inclined surfaces 45A, 45B, 63A, 63B, 65A and 65B increase the range in which a high-frequency current flows in sandwiched tissue. As a high-frequency current flows in a wide area in the tissue, a rapid increase of impedance in the sandwiched tissue can be suppressed. Thus, a sufficient amount of high-frequency current is applied to the sandwiched tissue, and performance of sealing or coagulating the tissue is improved.

In another treatment, in a state where living tissue is sandwiched between the jaws 21 and 22 as a treatment target, an operator causes the electric power supply apparatus 3 to output electric energy to the heat generator 41 by, for example, operating the operating apparatus 10. Thus, heat generated by the heat generator 41 is given as a treatment energy to the sandwiched tissue through the electrode 26. Then, the sandwiched tissue is incised by the heat from the heat generator 41 and a pressure from the projection 36 of the electrode 26. In the sandwiched tissue, a pressure acting at a site between the projection 36 and the abutting surface 56 is higher than pressures acting on other sites. For this reason, the tissue is incised at a site to which the projection 36 abutted, namely at a site upon which a high pressure acts, by giving the heat from the heat generator 41 to the tissue through the electrode 26. In the treatment for incising the tissue, the temperature of the heat generator 41 is higher compared to the foregoing treatment for sealing or coagulating tissue. In the treatment for incising the tissue, the temperature of the heat generator 41 is 250° C. or higher, and is adjusted so as to be at around 300° C., for example. Herein, tissue incised by heat generated by the heat generator 41 includes a solid organ such as a stomach wall, a vaginal wall, a liver, and the like, namely relatively thick tissue.

In the treatment for incising living tissue using the heat of the heat generator 41, a high-frequency electric power may be output from the electric power supply apparatus 3 to the electrodes 26 and 27 as described above, in addition to the output of the electric energy to the heat generator 41. Also in this case, the high-frequency current is applied to the sandwiched living tissue, and the tissue is sealed or coagulated by Joule heat caused by the high-frequency current. In other words, the tissue is sealed or coagulated at the same time as the incising of the tissue, by giving the high-frequency current to the sandwiched tissue in addition to the heat of the heat generator 41.

In the present embodiment, in the base 31 of the jaw 21, the extending surfaces 71A and 71B extend, from the electrode 26, outwardly in the width direction. And each of the extending surfaces 71A and 71B extends in such a manner that the surface is away from the virtual plane J as the surface becomes further away from the projection 36 (the projecting end T1) of the electrode 26 in the width direction of the jaw 21. In the present embodiment, in the base 51 of the jaw 22, the extending surfaces 72A and 72B extend, from the electrode 27 (a corresponding one of the electrode plates 57A and 57B), outwardly in the width direction. And each of the extending surfaces 72A and 72B extends in such a manner that the surface is away from the virtual plane J as the surface becomes further away from the abutting surface 56 (the projection 36 of the electrode 26) in the width direction of the jaw 22. For this reason, living tissue is sandwiched between the jaws 21 and 22, and even when the sandwiched tissue is abutted to the extending surfaces 71A, 71B, 72A, and 72B, the jaws 21 and 22 and the electrodes 26 and 27 are less susceptible to counterforce from the sandwiched tissue.

Through the provision of the extending surfaces 71A, 71B, 72A, and 72B, when the sandwiched tissue is abutted to the extending surfaces 71A, 71B, 72A, and 72B, a tension acts on the tissue, from each of the extending surfaces 71A, 71B, 72A, and 72B toward the side away from the projection 36 in the width direction of the jaws 21 and 22. In other words, the extending surfaces 71A, 71B, 72A, and 72B are a tension acting mechanism that cause the tensile action to act upon the tissue abutted to the surfaces, outwardly in the width directions of the jaws 21 and 22. For this reason, in a treatment for incising tissue, in a state where a tension acts upon the sandwiched tissue from the extending surfaces 71A, 71B, 72A, and 72B, which are a tension acting means, outwardly according to the width direction, the heat of the heat generator 41 is applied to the tissue through the electrode 26. Since the tension acts upon the sandwiched tissue outwardly in the width direction, from the extending surfaces 71A, 71B, 72B, and 72B, even when relatively thick tissue, such as a solid organ, is sandwiched between the jaws 21 and 22, the tissue is appropriately incised at a site to which the projection 36 (the projecting end T1) is abutted.

In the present embodiment, the inclined surfaces 45A and 45B extend in the electrode 26, and each of the inclined surfaces 45A and 45B is inclined in such a manner that it is separated from the jaw 22 as it becomes further away from the projection 36 (the projecting end T1) of the electrode 26 in the width direction of the jaw 21. Furthermore, in the present embodiment, the inclined surfaces 63A and 63B extend in the electrode 27, and each of the inclined surfaces 63A and 63B is inclined in such a manner that it is away from the jaw 21 as it becomes further away from the abutting surface 56 (the projection 36 of the electrode 26) in the width direction of the jaw 22. For this reason, in a state where living tissue is sandwiched between the jaws 21 and 22, the jaws 21 and 22 and the electrodes 26 and 27 are less susceptible to counterforce from the sandwiched tissue because of the inclined surfaces 45A, 45B, 63A, and 63B.

In the present embodiment, a tension acts on the tissue, from the inclined surfaces 45A, 45B, 63A, and 63B, in addition to the extending surfaces 71A, 71B, 72A, and 72B, to the side away from the projection 36 in the width direction of the jaws 21 and 22. For this reason, when relatively thick tissue, such as a solid organ, is incised, the tissue is further incised as appropriate at a site to which the projection 36 (the projecting end T1) is abutted, and the performance of incision is improved. As described above, in the present embodiment, the treatment instrument 2 that appropriately incises tissue sandwiched between a pair of jaws 21 and 22 is provided.

In a configuration in which the end effector 8 is bendable with respect to the shaft 6, the larger the bending angle of the end effector 8 is with respect to the shaft 6, the smaller the grasping force between the jaws 21 and 22 tends to be. In the present embodiment, a tension acts on the tissue, from the inclined surfaces 45A, 45B, 63A, and 63B, in addition to the extending surfaces 71A, 71B, 72A, and 72B, to the side away from the projection 36 in the width direction of the jaws 21 and 22. For this reason, even when the end effector 8 is greatly bent with respect to the shaft 6, the tissue is appropriately incised at a site to which the projection 36 (the projecting end T1) comes into contact due to a tension from the extending surfaces 71A, 71B, 72A, and 72B, and the inclined surfaces 45A, 45B, 63A, and 63B. In other words, even if the grasping force between the jaws 21 and 22 decreases as compare with a case in which the end effector 8 is straight with respect to the shaft 6, the sandwiched tissue is appropriately incised.

In the present embodiment, each of the extending surfaces 71A and 71B continues to a corresponding one of the outer edge surfaces 67A and 67B of the base 31, and extends to the outer edge of the jaw 21 in the width direction. Each of the extending surfaces 72A and 72B continues to a corresponding one of the outer edge surfaces 68A and 68B of the base 51, and extends to the outer edge of the jaw 22 in the width direction. Furthermore, each of the extending surfaces 71A, 71B, 72A, and 72B is made of a resin, etc., and has a low thermal conduction rate. For this reason, when a treatment is conducted as described above, heat such as Joule heat due to a high-frequency current and heat of the heat generator 41, etc., is less invasive from a site at which the jaws 21 and 22 sandwiches the tissue to the outside of the width direction of the jaws 21 and 22. In other words, in a treatment, outward thermal invasion in the width direction from the site at which the tissue is sandwiched is reduced. The lateral thermal invasion, which is outward thermal invasion in the width direction of the jaws 21 and 22, is reduced, and thereby the performance of treatment is improved.

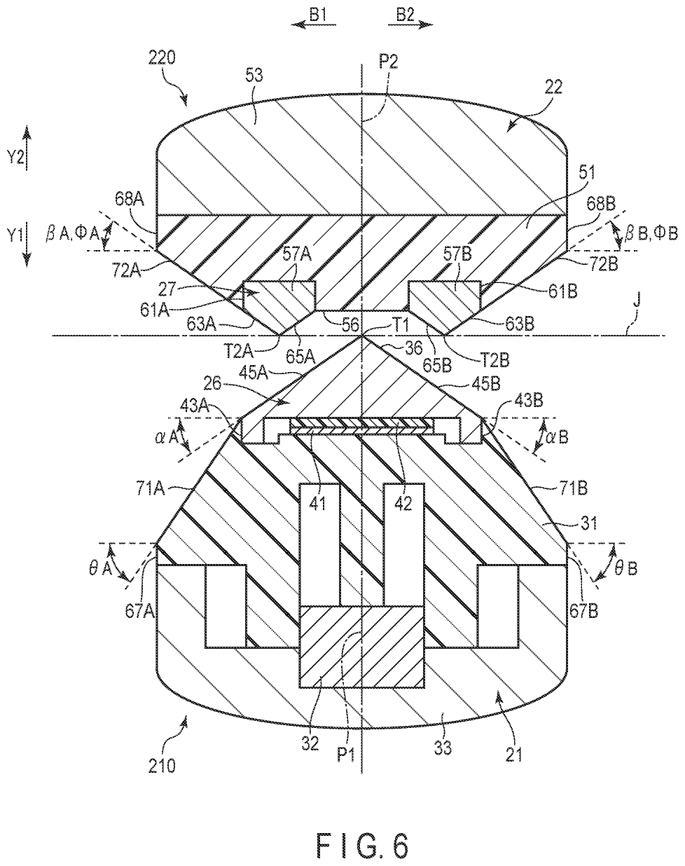

The above exemplary embodiment can be modified as shown in FIG. 6, in which the acute angle αA relative to virtual plane J of the inclined surface 45A of the electrode 26 is an angle differing from the acute angle θA relative to the virtual plane J of the extending surface 71A of the base 31. The acute angle αB relative to virtual plane J of the inclined surface 45B of the electrode 26 is an angle differing from the acute angle θB relative to the virtual plane J of the extending surface 71B of the base 31. Herein, in the example shown in FIG. 6, the acute angles θA and θB are larger than the acute angles αA and αB, respectively. However, the acute angles θA and θB may be smaller than acute angles αA and αB, respectively.

In this modified example, the acute angle βA relative to virtual plane J of the inclined surface 63A of the electrode 27 is the same or substantially the same angle as the acute angle φA relative to the virtual plane J of the extending surface 72A of the base 51. Then, the acute angle βB relative to virtual plane J of the inclined surface 63B of the electrode 27 is the same or substantially the same angle as the acute angle φB relative to the virtual plane J of the extending surface 72B of the base 51. Furthermore, in the example shown in FIG. 6, the acute angles φA and φB are larger than the acute angles αA and αB, respectively. Thus, the operations and advantageous effects similar to those of the above embodiment, etc. are achieved in the present modification.

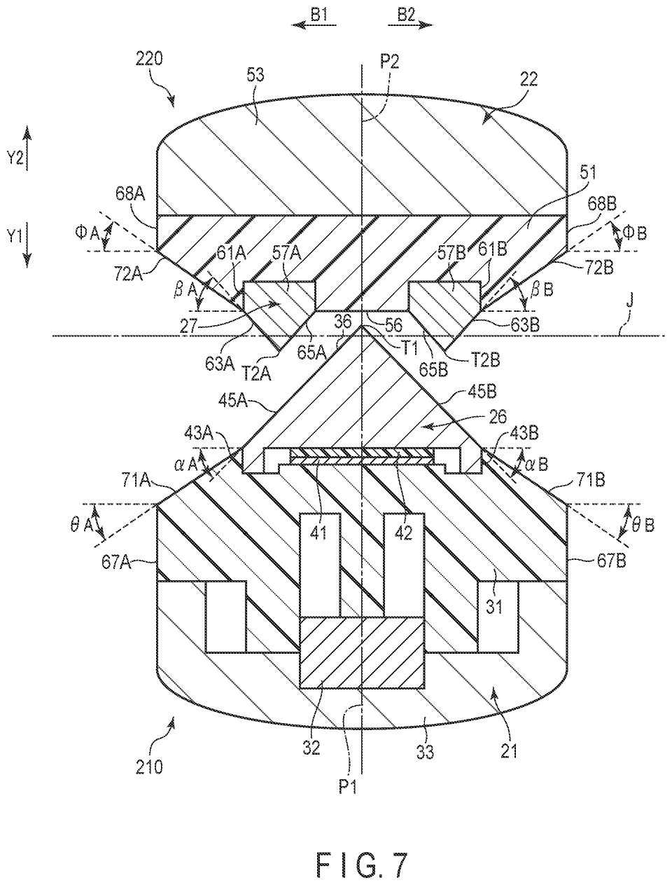

Even in another modified example shown in FIG. 7, the acute angle αA of the inclined surface 45A and the acute angle αB of the inclined surface 45B are angles differing from the acute angle θA of the extending surface 71A and the acute angle θB of the extending surface 71B, respectively. In the example shown in FIG. 7, the acute angles θA and θB are smaller than the acute angles αA and αB, respectively. However, the acute angles θA and θB may be larger than acute angles αA and αB, respectively.

In the present modification, the acute angle βA relative to virtual plane J of the inclined surface 63A of the electrode 27 is an angle differing from the acute angle φA relative to the virtual plane J of the extending surface 72A of the base 51. The acute angle βB relative to virtual plane J of the inclined surface 63B of the electrode 27 is an angle differing from the acute angle φB relative to the virtual plane J of the extending surface 72B of the base 51. In the example shown in FIG. 7, the acute angles φA and φB are smaller than the acute angles βA and βB, respectively. However, the acute angles φA and φB may be larger than acute angles SA and SB, respectively. Thus, the operations and advantageous effects similar to those of the above embodiment, etc. are achieved in the present modification.

In a modification, the acute angles φA and φB are angles differing from the acute angles βA and βB, respectively. The acute angles αA and αB are the same or substantially the same as the acute angles θA and θB, respectively. Also in this modification, the operations and advantageous effects similar to those of the above embodiment, etc. are achieved.

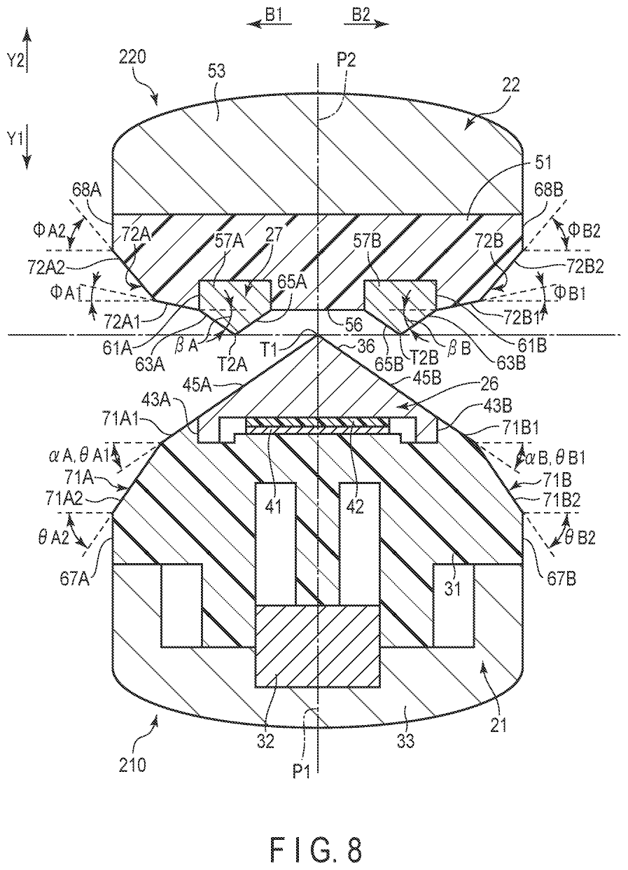

In another modification shown in FIG. 8, the extending surface (first extending surface) 71A of the base (first base) 31 includes inclined surfaces 71A1 and 71A2, and the extending surface (third extending surface) 71B of the base 31 includes inclined surfaces 71B1 and 71B2. The inclined surface 71A1 extends outwardly from the outer edge surface (first outer edge surface) 43A of the electrode (first electrode) 26 in respect to the width direction, and the extending surface 71A2 is adjacent to the outer side of the inclined surface 71A1 in the width direction. The inclined surface 71A2 extends outwardly in the width direction, up to the outer edge surface 67A of the base 31. The inclined surface 71B1 extends outwardly from the outer edge surface (third outer edge surface) 43B of the electrode (first electrode) 26 in the width direction, and the extending surface 71B2 is adjacent to the outer side of the inclined surface 71B1 according to the width direction. Then, the inclined surface 71B2 extends outwardly in the width direction, up to the outer edge surface 67B of the base 31.

Also in the present modification, each of the extending surfaces 71A or 71B extends in such a manner that the surface is away from the virtual plane J as the surface becomes further away from the projection 36 (the projection end T1) of the electrode 26 in the width direction of the jaw 21. Accordingly, each of the extending surfaces 71A1, 71A2, 71B1, and 71B2 is inclined to the virtual plane J in such a manner that the surface is separated from the jaw 22 as the surface becomes more outwardly according to the width direction of the jaw 21. In the present modification, the inclined surfaces 71A1 and 71B1 are arranged symmetrically or substantially symmetrically to each other with respect to the virtual central plane P1. The inclined surfaces 71A2 and 71B2 are arranged symmetrically or substantially symmetrically to each other with respect to the virtual central plane P1.

The acute angle θA1 relative to virtual plane J of the inclined surface 71A1 is an angle differing from the acute angle θA2 relative to the virtual plane J of the extending surface 71A2. The acute angle θB1 relative to virtual plane J of the inclined surface 71B1 is an angle differing from the acute angle θB2 relative to the virtual plane J of the extending surface 71B2. In the present embodiment, the acute angle θA1 of the inclined surface 71A1 and the acute angle θB1 of the inclined surface 71B1 are the same or substantially the same, and the acute angle θA2 of the inclined surface 71A2 and the acute angle θB2 of the inclined surface 71B2 are the same or substantially the same. And the acute angle θA1 of the inclined surface 71A1 and the acute angle θB1 of the inclined surface 71B1 are the same or substantially the same as the acute angle αA of the inclined surface 45A and the acute angle αB of the inclined surface 45B of the electrode 26, respectively. However, the acute angle θA1 of the inclined surface 71A1 and the acute angle θB1 of the inclined surface 71B1 are angles differing from the acute angle αA of the inclined surface 45A and the acute angle αB of the inclined surface 45B of the electrode 26, respectively. In this case, the acute angle θA2 of the inclined surface 71A2 and the acute angle θB2 of the inclined surface 71B2 may be the same or substantially the same as the acute angle αA of the inclined surface 45A and the acute angle αB of the inclined surface 45B of the electrode 26, respectively.

In the present modification (see FIG. 8), the extending surface (second extending surface) 72A of the base (second base) 51 includes inclined surfaces 72A1 and 72A2, and the extending surface (fourth extending surface) 72B of the base 51 includes inclined surfaces 72B1 and 72B2. The inclined surface 72A1 extends outwardly in the width direction from the outer edge surface (second outer edge surface) 61A of the electrode (second electrode) 27, and the inclined surface 72A2 is adjacent to the outer side of the inclined surface 72A1 in the width direction. And the inclined surface 72A2 extends outwardly in the width direction, up to the outer edge surface 68A of the base 51. The inclined surface 72B1 extends outwardly in the width direction from the outer edge surface (fourth outer edge surface) 61B of the electrode (second electrode) 27, and the inclined surface 72B2 is adjacent to the outer side of the inclined surface 72B1 according to the width direction. And the inclined surface 72B2 extends outwardly in the width direction, up to the outer edge surface 68B of the base 51.

Also in the present modification, each of the extending surfaces 72A and 72B extends in such a manner that the surface is away from the virtual plane J as the surface becomes further away from the abutting surface 56 (the projection 36 of the electrode 26) in the width direction of the jaw 22. Accordingly, each of the extending surfaces 72A1, 72A2, 72B1, and 72B2 is inclined to the virtual plane J in such a manner that the surface is separated from the jaw 21 as the surface becomes further outward according to the width direction of the jaw 22. In the present modification, the inclined surfaces 72A1 and 72B1 are arranged symmetrically or substantially symmetrically to each other with respect to the virtual central plane P2. And the inclined surfaces 72A2 and 72B2 are arranged symmetrically or substantially symmetrically to each other with respect to the virtual central plane P2.

The acute angle φA1 relative to virtual plane J of the inclined surface 72A1 is an angle differing from the acute angle φA2 relative to the virtual plane J of the inclined surface 72A2. And the acute angle φB1 relative to virtual plane J of the inclined surface 72B1 is an angle differing from the acute angle φB2 relative to the virtual plane J of the extending surface 72B2. In the present modification, the acute angle φA1 of the inclined surface 72A1 and the acute angle φB1 of the inclined surface 72B1 are the same or substantially the same, and the acute angle φA2 of the inclined surface 72A2 and the acute angle φB2 of the inclined surface 72B2 are the same or substantially the same. And each of the acute angle φA1 of the inclined surface 72A1, the acute angle φB1 of the inclined surface 72B1, the acute angle φA2 of the inclined surface 72A2, and the acute angle φB2 of the inclined surface 72B2 is an angle different from the acute angle αA of the inclined surface 45A and the acute angle αB of the inclined surface 45B of the electrode 26. However, the acute angle φA1 of the inclined surface 72A1 and the acute angle φB1 of the inclined surface 72B1 may be the same or substantially the same as the acute angle αA of the inclined surface 45A and the acute angle αB of the inclined surface 45B of the electrode 26, respectively. The acute angle φA2 of the inclined surface 72A2 and the acute angle φB2 of the inclined surface 72B2 may be the same or substantially the same as the acute angle αA of the inclined surface 45A and the acute angle αB of the inclined surface 45B of the electrode 26, respectively. Thus, the operations and advantageous effects similar to those of the above embodiment, etc. are achieved in the present modification.

In a modification, each of the extending surfaces 71A and 71B has three or more inclined surfaces having mutually different angles with respect to the virtual surface J. Similarly, each of the extending surfaces 72A and 72B has three or more inclined surfaces having mutually different angles with respect to the virtual surface J. In another modification, each of the extending surfaces 71A and 71B has multiple inclined surfaces having mutually different angles with respect to the virtual surface J, and each of the extending surfaces 72A and 72B is constituted by a single inclined surface. In another modification, each of the extending surfaces 72A and 72B has multiple inclined surfaces having mutually different angles with respect to the virtual surface J, and each of the extending surfaces 71A and 71B is constituted by a single inclined surface. Accordingly, the operations and advantageous effects similar to those of the foregoing embodiments, etc. are achieved in these modifications.

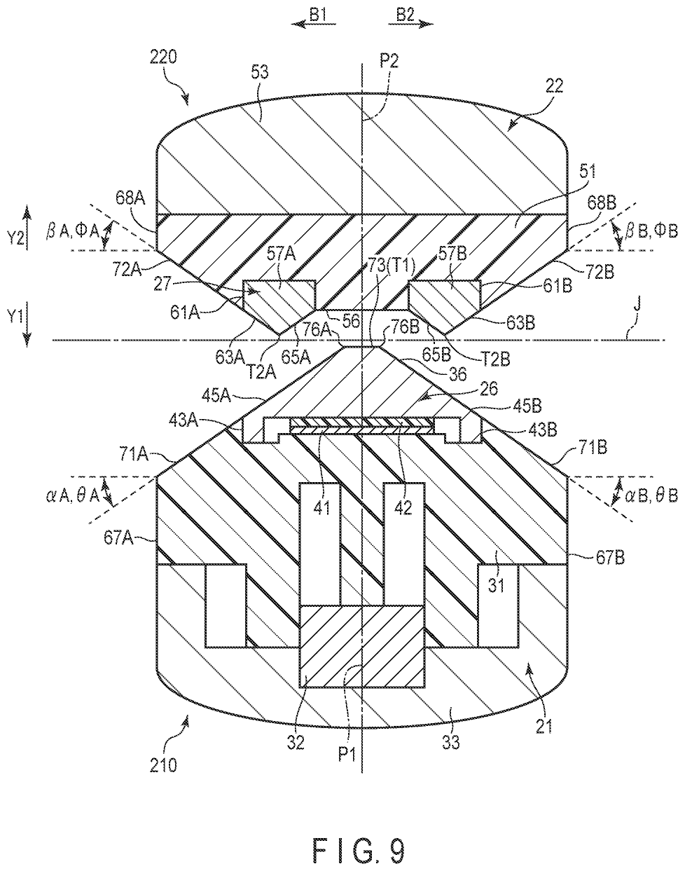

In another modification shown in FIG. 9, the projection 36 of the electrode (first electrode 26) includes a projecting end surface 73. The projecting end surface 73 constitutes the projecting end T1 of the projection 36, and faces the abutting surface 56 of the jaw 22. The projecting end surface 73 is formed in a plane or substantially a plane, for example. The projecting end surface 73 is abuttable to the abutting surface 56 when the jaws 21 and 22 are closed on each other. The projecting end surface 73 intersects with (is perpendicular or substantially perpendicular to) the opening and closing directions of the jaw 21. The virtual central plane P1 of the jaw 21 passes the projecting end surface 73, and the projecting end surface 73 is formed symmetrically or substantially symmetrically with respect to the virtual central plane P1. In another modification, the projecting end surface 73 may be formed in a curved surface, such as an R surface.

In this modification (FIG. 9), each of the inclined surfaces 45A and 45B is adjacent to the outer side of the projecting end surface 73 in the width direction of the jaw 21. Then, the projecting end surface 73 continuously extends between the inclined surfaces 45A and 45B. Each of the inclined surfaces 45A and 45B is inclined with respect to the projecting end surface 73, in such a manner that the surface is spaced from the jaw 22 as the surface becomes distant from the projecting end surface 73 in the width direction. In the electrode 26, the edge 76A is formed at a location where the projecting end surface 73 intersects with the inclined surface 45A. And the edge 76B is formed at a location where the projecting end surface 73 intersects with the inclined surface 45B.

Thus, the operations and advantageous effects similar to those of the above exemplary embodiment, etc. are achieved in the present modification. In the present modification, the projecting end T1 of the projection 36 is constituted by the projecting end surface 73, and is formed in a plane shape. For this reason, in a treatment for sealing or coagulating living tissue sandwiched between the jaws 21 and 22, a pressure acting on the tissue from the projection 36 does not become excessively large. Therefore, in a treatment for sealing or coagulating living tissue such as blood vessels, etc., unintentional incision of sandwiched tissue by the projection 36 can be effectively prevented.

In the present embodiment, even if the projecting end surface 73 is provided, the edges (edge lines) 76A and 76B are formed in the projection 36 of the electrode 26. For this reason, in a treatment for incising living tissue sandwiched between the jaws 21 and 22, a pressure of an appropriate amplitude acts on the sandwiched tissue from the projection 36. Therefore, in a treatment for incising living tissue such as a solid organ, etc., the sandwiched tissue is appropriately incised at a site to which the projection 36 is abutted.

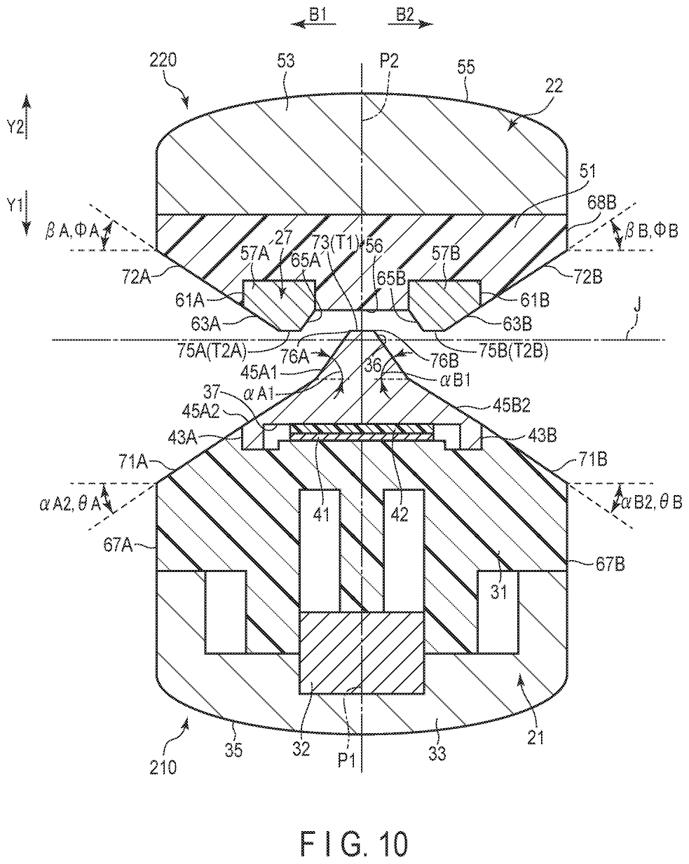

In another modification shown in FIG. 10, similarly to the above modification shown in FIG. 9, the projecting end surface 73 is provided in the projection 36 of the electrode (first electrode) 26. In the present modification, however, in the electrode 26, two inclined surfaces 45A1 and 45A2 are provided instead of the inclined surface 45A, and two inclined surfaces 45B1 and 45B2 are provided instead of the inclined surface 45B. In the present modification, each of the inclined surfaces (first inclined surfaces) 45A1 and 45B1 is adjacent to the outer side of the projecting end surface 73 in the width direction of the jaw 21. Then, the projecting end surface 73 continuously extends between the inclined surfaces 45A1 and 45B1. Each of the inclined surfaces 45A1 and 45B1 is inclined with respect to the projecting end surface 73, in such a manner that the surface is away from the jaw 22 as it becomes distant from the projecting end surface 73 according to the width direction.

The inclined surface (second inclined surface) 45A2 is adjacent to the outer side of the inclined surface 45A1 in the width direction of the jaw 21. The inclined surface 45A2 continuously extends between the inclined surface 45A1 and the extending surface 71A of the base 31. Thus, the inclined surface 45A2 continues toward the outer side of the width direction, up to the extending surface 71A of the base 31, namely the outer edge surface 43A of the electrode 26. The inclined surface (second inclined surface) 45B2 is adjacent to the outer side of the inclined end surface 45B1 in the width direction of the jaw 21. The inclined surface 45B2 continuously extends between the inclined surface 45B1 and the extending surface 71B of the base 31. Thus, the inclined surface 45B2 continues toward the outer side of the width direction, up to the extending surface 71B of the base 31, namely the outer edge surface 43B of the electrode 26. Each of the inclined surfaces 45A2 and 45B2 is inclined with respect to the projecting end surface 73, in such a manner that the surface is away from the jaw 22 as the surface becomes distant from the projecting end surface 73 in the width direction.

In the present modification, the inclined surfaces 45A1 and 45B1 are arranged symmetrically or substantially symmetrically to each other with respect to the virtual central plane P1. Then, the acute angle αA1 relative to the projecting end surface 73 (the virtual plane J) of the inclined surface 45A1 is the same or substantially the same angle as the acute angle αB1 relative to the projecting end surface 73 (the virtual plane J) of the inclined surface 45B1. In the present modification, the inclined surfaces 45A2 and 45B2 are arranged symmetrically or substantially symmetrically to each other with respect to the virtual central plane P1. Then, the acute angle αA2 relative to the projecting end surface 73 (the virtual plane J)) of the inclined surface 45A2 is the same or substantially the same angle as the acute angle αB2 relative to the projecting end surface 73 (the virtual plane J) of the inclined surface 45B2.

In the present modification, the acute angle αA1 of the inclined surface 45A1 and the acute angle αB1 of the inclined surface 45B1 are larger than the acute angle αA2 of the inclined surface 45A2 and the acute angle αB2 of the inclined surface 45B2, respectively. The acute angle αA2 of the inclined surface 45A2 and the acute angle αB2 of the inclined surface 45B2 are the same angles as the acute angle θA of the extending surface 71A and the acute angle θB of the extending surface 71B of the base 31, respectively. However, the acute angle αA2 of the inclined surface 45A2 and the acute angle αB2 of the inclined surface 45B2 may be angles differing from the acute angle θA of the extending surface 71A and the acute angle θB of the extending surface 71B of the base 31, respectively.

In the present modification, in the electrode (second electrode) 27, the electrode plate 57A includes a projecting end surface 75A, and the electrode plate 57B includes the projecting end surface 75B. The projecting end surface 75A constitutes the projecting end T2A of the electrode plate 57A, and faces the inclined surface 45A2 of the electrode 26. And the projecting end surface 75B constitutes the projecting end T2B of the electrode plate 57B, and faces the inclined surface 45B2 of the electrode 26. Each of the projecting end surfaces 75A and 75B is formed in a plane or substantially a plane, for example. Each of the projecting end surfaces 75A and 75B intersects with (is perpendicular or substantially perpendicular to) the opening and closing directions of the jaw 21. The projecting end surfaces 75A and 75B are arranged symmetrically or substantially symmetrically to each other with respect to the virtual central plane P2. The projecting end surface 75A continuously extends between the inclined surfaces 63A and 65A in the electrode plate 57A. Then, the projecting end surface 75B continuously extends between the inclined surfaces 63B and 65B in the electrode plate 57B.

Accordingly, operations and advantageous effects similar to those of the above modification (FIG. 9), etc. are achieved in the present modification. In the present modification, the acute angle αA1 of the inclined surface 45A1 and the acute angle αB1 of the inclined surface 45B1 are larger than the acute angle αA2 of the inclined surface 45A2 and the acute angle αB2 of the inclined surface 45B2, respectively. If each of the acute angle αA1 of the inclined surface 45A1 and the acute angle αB1 of the inclined surface 45B1 is large, a length of projection of the projection 36 of the electrode 26 toward the jaw 22 is large. In addition, if the length of projection of the projection 36 is large, a pressure acting on sandwiched tissue from the projection 36 also becomes large. Thus, the performance of incision improves in a treatment for incising living tissue, such as a solid organ, etc.

In the present modification, since each of the acute angle αA2 of the inclined surface 45A2 and the acute angle αB2 of the inclined surface 45B2 is small, it becomes possible to secure a long distance from the electrode back surface 37 to which the heat generator 41 is attached to the jaw back surface 35. As the distance from the electrode back surface 37 to the jaw back surface 35 becomes large, it becomes difficult for heat, etc. of the heat generator 41 to be transmitted to the jaw back surface 35, and the operability in a body cavity, etc. for an operator is improved.

Furthermore, in the present modification, the projecting end surfaces 75A and 75B are provided in the electrode 27, and each of the projecting end T2A of the electrode plate 57A and the projecting end T2B of the electrode plate 57B is formed in a plate shape. For this reason, when a treatment is performed with living tissue being sandwiched between the jaws 21 and 22, a pressure acting on the tissue from the electrode plates 57A and 57b does not become excessively large. Therefore, unintentional damaging of the sandwiched tissue by the electrode plates 57A and 57B can be effectively prevented in a treatment.

In another modification in which the projecting end surface 73 is not provided in the electrode 26 similarly to the above embodiment (FIGS. 4 and 5), the inclined surfaces 45A1, 45A2, 45B1, and 45B2 may be provided in the electrode 26, similarly to the fifth modification. In this case, the location where the inclined surfaces 45A1 and 45B1 intersect with each other is the projecting end T1 of the electrode 26.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment instrument comprising:
a pair of jaws including a first jaw and a second jaw that are closable and openable with respect to each other and are arranged with a virtual plane interposed therebetween in a closing and opening direction, the virtual plane extending along a longitudinal direction and a width direction of the pair of jaws so as to be transverse to the closing and opening direction;
a first electrode that is provided on the first jaw and is openable and closable together with the first jaw with respect to the second jaw, the first electrode including a projection projecting toward the second jaw, and a first outer edge surface forming an outer edge of the first electrode in the width direction of the pair of jaws, and the first electrode being configured to incise tissue sandwiched between the pair of jaws by the projection;
a first base provided in the first jaw and attached to a back surface of the first electrode, the first base having a lower heat conduction rate than that of the first electrode, the first base including a first extending surface that extends from the first outer edge surface of the first electrode toward a side away from the projection in the width direction so as to slope away from the virtual plane in the closing and opening direction, and the first extending surface in the first base is configured to abut living tissue sandwiched between the pair of jaws;
a second electrode that is provided on the second jaw and is openable and closable together with the second jaw with respect to the first jaw, the second electrode including a second outer edge surface forming an outer edge of the second electrode in the width direction; and
a second base provided in the second jaw and attached to a back surface of the second electrode, the second base having a lower heat conduction rate than that of the second electrode, the second base including a second extending surface that extends from the second outer edge surface of the second electrode toward a side away from the projection in the width direction so as to slope away from the virtual plane in the closing and opening direction, and the second extending surface in the second base is configured to abut living tissue sandwiched between the pair of jaws.

2. The treatment instrument according to claim 1, wherein each of the first extending surface and the second extending surface extends in a plane.

3. The treatment instrument according to claim 1,
wherein the first extending surface and the second extending surface face each other.

4. The treatment instrument according to claim 1, wherein
the second base includes an abutting surface on which the projection of the first electrode is abuttable, and
the second electrode is attached to the second base between the second extending surface and the abutting surface.

5. The treatment instrument according to claim 4, wherein the first electrode is spaced from the second electrode in a state in which the projection abuts the abutting surface of the second base.

6. The treatment instrument according to claim 4, wherein the second electrode projects toward the first jaw from the abutting surface of the second base.

7. The treatment instrument according to claim 4, wherein the second electrode includes a pair of electrode plates arranged with the abutting surface interposed therebetween.

8. The treatment instrument according to claim 1, wherein:
the first electrode includes a third outer edge surface that forms an outer edge of the first electrode on an opposite side of the first outer edge surface in the width direction,
the first base includes a third extending surface that extends from the third outer edge surface of the first electrode toward a side away from the projection in the width direction so as to slope away from the virtual plane in the closing and opening direction, the third extending surface being configured to abut living tissue sandwiched between the pair of jaws,
the second electrode includes a fourth outer edge surface that forms an outer edge of the second electrode on an opposite side of the second outer edge surface in the width direction,
the second base includes a fourth extending surface that extends from the fourth outer edge surface of the second electrode toward a side away from the projection in the width direction so as to slope away from the virtual plane in the closing and opening direction, the fourth extending surface being configured to abut living tissue sandwiched between the pair of jaws,
the third extending surface and the first extending surface are arranged symmetrically with respect to a second virtual plane that is orthogonal to the virtual plane and extends through the projection of the first electrode, and
the fourth extending surface and the second extending surface are arranged symmetrically with respect to the second virtual plane.

9. The treatment instrument according to claim 1, wherein the projection of the first electrode includes a projecting end surface extending in a direction that intersects the opening and closing direction of the pair of jaws.

10. The treatment instrument according to claim 9, wherein
the first electrode includes:
a first inclined surface extending from the projecting end surface toward the first extending surface of the first base, the first inclined surface extending away from the projecting end surface in the width direction so as to be inclined in a direction away from the second jaw; and
a second inclined surface continuously extending from the first inclined surface to the first extending surface of the first base, the second inclined surface extending away from the projecting end surface in the width direction so as to be inclined in a direction away from the second jaw.

11. The treatment instrument according to claim 9, wherein
the first electrode includes:
an inclined surface extending from the first projecting end surface toward the first extending surface, the inclined surface extending away from the projecting end surface in the width direction so as to be inclined in a direction away from the second jaw; and
an edge formed at a location where the projecting end surface intersects the inclined surface.

12. The treatment instrument according to claim 1, further comprising:
a shaft extending along an axial direction, a distal end portion of the shaft being coupled to an end effector comprising the pair of jaws, the first electrode, and the second electrode; and
a driving member extending in the axial direction of the shaft, the driving member being configured to open or close the pair of jaws with respect to each other by moving along the axial direction of the shaft, wherein:
the end effector is configured to swivel with respect to the shaft around a location where the end effector is coupled to the shaft.

13. The treatment instrument according to claim 12, further comprising a link mechanism that connects the driving member to the end effector,
wherein the shaft is coupled to the end effector at a position overlapping the link mechanism in a radial direction of the shaft transverse to the axial direction.

14. The treatment instrument according to claim 1, further comprising a heat generator provided on the back surface of the first electrode.

15. The treatment instrument according to claim 14, wherein:
the first electrode and the second electrode are configured to receive electric energy and apply a high-frequency current to living tissue sandwiched between the pair of jaws, and
the heat generator is configured to generate heat through electric energy supplied thereto.

16. A method of using the treatment instrument according to claim 1, the method comprising:
closing the pair of jaws to sandwich living tissue between the pair of jaws such that:
the living tissue abuts the first electrode, the second electrode, the first extending surface of the first base, and the second extending surface of the second base; and
the first extending surface and the second extending surface exert a tensile force on the living tissue outwardly in in the width direction of the pair of jaws; and
applying treatment energy to the living tissue via at least one of the first electrode and the second electrode and incising the living tissue at a site abutting the projection in a state in which the tensile force is exerted upon the living tissue.

17. A treatment instrument comprising:
a pair of jaw assemblies including a first jaw assembly and a second jaw assembly relatively openable and closable with respect to the first jaw assembly, a virtual plane that: (i) extends along a longitudinal direction and a width direction of the pair of jaw assemblies, (ii) intersects an opening and closing direction of the pair of jaw assemblies, and (iii) is located at a position where the first jaw assembly and the second jaw assembly meet each other in a closed state, wherein:
the first jaw assembly includes:
a first electrode including a projection projecting toward the second jaw assembly, and a first outer edge surface that forms an outer edge of the first electrode in the width direction of the pair of jaw assemblies, the first electrode being configured to incise living tissue sandwiched between the pair of jaw assemblies; and
a first base including a first extending surface having a lower heat conduction rate than that of the first electrode, the first extending surface being configured to abut living tissue sandwiched between the pair of jaw assemblies;
the second jaw assembly includes:
a second electrode including a second outer edge surface that forms an outer edge of the second electrode in the width direction; and
a second base including a second extending surface having a lower heat conduction rate than that of the second electrode, the second extending surface being configured to abut living tissue sandwiched between the pair of jaw assemblies;
the first extending surface extends from the first outer edge surface of the first electrode toward a side away from the projection in the width direction so as to slope away from the virtual surface in the opening and closing direction, and
the second extending surface extends from the second outer edge surface of the second electrode in the second base toward a side away from the projection in the width direction so as to slope away from the virtual surface in the opening and closing direction.
